(19) United States
(12) Patent Application Publication
(10) Pub. No.: US 2011/0319854 A1
(43) Pub. Date: Dec. 29, 2011

(54) DISPOSABLE BODILY FLUID HANDLING COMPOSITE PAD

(75) Inventors: Ayami Suga, Kagawa (JP); Yusuke Kosako, Kagawa (JP)

(73) Assignee: UNI-CHARM CORPORATION, SHIKOKUCHUO-SHI, EHIME (JP)

(21) Appl. No.: 12/525,040

(22) PCT Filed: Jul. 28, 2009
(86) PCT No.: PCT/JP09/63444

§ 371 Date: Dec. 9, 2009

(51) Int. Cl. A61F 13/47

(52) U.S. Cl. 604/385.14

(57) ABSTRACT

The present invention aims to provide a disposable bodily fluid handling composite pad improved so that, when it is desired to peel off each of component pads from the composite pad, an appropriate position of the composite pad to be pinched by the finger can be easily recognized. A menstruation napkin 1 as a typical example of the disposable bodily fluid handling composite pad comprises two or more component pads laminated one on another. These component pads are temporarily joined together along temporarily joined regions. A first component pad 10 is formed with substantially continuous temporarily joined region 13 which is interrupted by a non-joined region on the upper portion of the accompanying drawings. In the non-joined region 14, a graphic of heart mark is displayed as an indicator element 61 so as to be visually recognized through a liquid-absorbent sheet 11.

### TECHNICAL FIELD

The present invention generally relates to a disposable bodily fluid handling composite pad and more particularly to such a composite pad used in the form of, for example, a menstruation napkin, a urine absorbent pad or panty liner.

### RELATED ART

It is known to laminate two or more component pads one upon another and thereby to obtain a disposable bodily fluid handling composite pad. For example, PATENT CITATION 1 discloses such composite pad comprising laminated two or more liquid-absorbent component pads temporarily joined together along transversely opposite side edges by locally crimping these laminated component pads. It is possible for the wearer of such composite pad to peel off the component pad facing the wearer's body from the composite pad one by one. More specifically, when the component pad contacting the wearer's skin is getting soiled, it may be peeled off from the remaining components to expose the adjacent fresh component pad for use.

### PRIOR ART DOCUMENT

### Patent Citation

PATENT CITATION 1: JP 4226086 B2

### SUMMARY OF THE INVENTION

### Problem to be Solved by the Invention

This composite pad of prior art is designed so that the soiled component pad is peeled off from the composite pad by pinching the end portion thereof with the fingers and pull it off from the composite pad. To assure the soiled component pad to be smoothly peeled off from the composite pad, it is required for the wearer to pinch non-crimped region of the composite pad because it will be difficult to peel off the soiled component pad starting from the crimped region. However, the composite pad is usually of the same color over all and it is difficult for the wearer to catch sight of the non-crimped region in a moment.

In view of the problem as has been described above, it is an object of the present invention to provide a disposable bodily fluid handling composite pad improved so that the appropriate region to be pinched with the fingers can be easily recognized when it is desired to peeled off the soiled component pad from the composite pad.

### Measure to Solve the Problem

The present invention relates to an improvement of a disposable bodily fluid handling composite pad having a longitudinal direction, a transverse direction, an upper side facing a wearer's body and a lower side opposite to the upper side, and comprising component pads each including an upper liquid-absorbent sheet and a lower liquid-impervious sheet wherein two or more of the component pads are laminated one on another.

The disposable bodily fluid handling composite pad is characterized in that:

the two or more component pads respectively include temporarily joined regions along which the component pads are temporarily joined to each other so as to be peeled off from other component pads and non-joined regions; and

each of the component pads are formed with indicator elements adapted to be visually recognized through the liquid-absorbent sheet and the indicator elements are formed in the non-joined regions defined along peripheral edges of the component pads and adapted to indicate at least partially the non-joined regions.

According to one preferred embodiment of the present invention, the temporally joined regions are formed substantially in continuous fashion along peripheral edges of the component pads and the non-joined regions are defined along partial segments of the peripheral edges so as to interrupt the substantially continuous temporally joined regions.

According to another preferred embodiment of the present invention, the indicator element comprises at least any one of letter, symbol and graphic displayed in the non-joined regions.

According to another preferred embodiment of the present invention, the indicator element comprises at least any one of letter, symbol and graphic serving to indicate the non-joined regions.

According to another preferred embodiment of the present invention, the indicator element is defined by coloring of the non-joined regions.

According to another preferred embodiment of the present invention, the indicator element comprises cutouts formed by partially cutting away the component pads.

### Effect of the Invention

Two or more liquid-absorbent component pads laminated one on another respectively have temporarily joined regions along which these component pads are detachably and temporarily joined together and non-joined regions. The respective component pads are formed with indicator elements adapted to be visually recognized through the associated liquid-absorbent sheets and to indicate the non-joined regions. In this way, the appropriate position of the composite pad to be pinched with the fingers can be easily recognized as instructed by the indicator element.

### BRIEF DESCRIPTION OF THE DRAWINGS

FIG. 1 is a perspective view of a menstruation napkin.

FIG. 2 is a sectional view taken along the line II-II in FIG. 1.

FIG. 3 is a schematic diagram illustrating Example 1.

FIG. 4 is a schematic diagram illustrating Example 2.

FIG. 5 is a schematic diagram illustrating Example 3.

FIG. 6 is a schematic diagram illustrating Example 4.

FIG. 7 is a schematic diagram illustrating Example 5.

FIG. 8 is a schematic diagram illustrating Example 6.

FIG. 9 is a schematic diagram illustrating Example 7.

### IDENTIFICATION OF REFERENCE NUMERALS USED IN THE DRAWINGS

1 menstruation napkin (disposable body fluid handling composite pad)
10 first pad (pad)
11 liquid-absorbent sheet
12 liquid-impervious sheet
13 temporarily joined region
14 non-joined region
20 second pad (pad)
21 liquid-absorbent sheet
22 liquid-impervious sheet
23 temporarily joined region
24 non-joined region
30 third pad (pad)
31 liquid-absorbent sheet
32 liquid-impervious sheet
33 temporarily joined region
34 non-joined region
61-63 indicator elements
65-67 indicator elements
68 cutout
69 colored region
70 cutout
71 colored region
X transverse direction
Y longitudinal direction

### DETAILED DESCRIPTION OF THE PREFERRED EMBODIMENTS

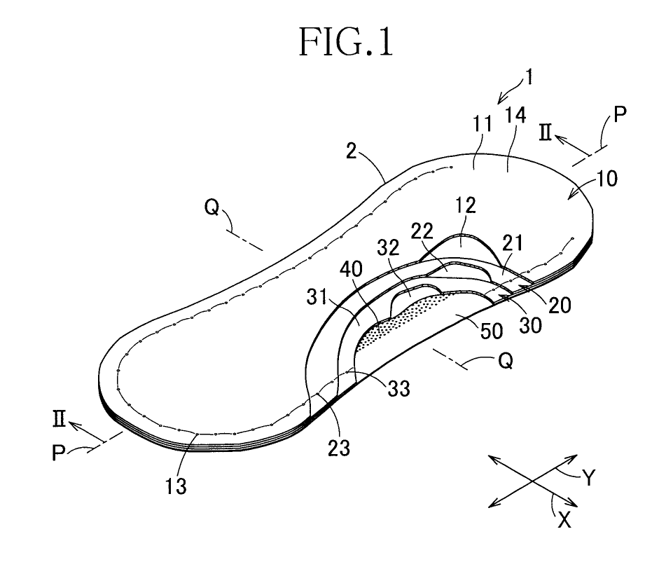

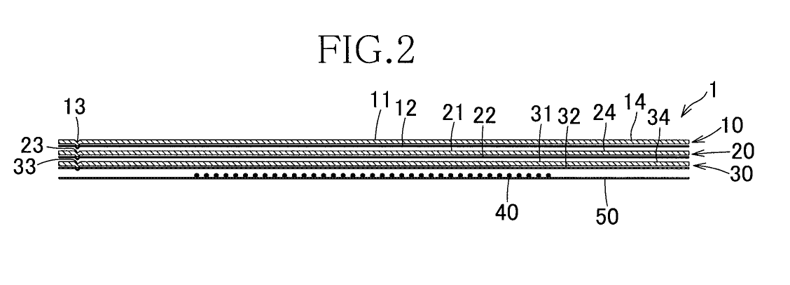

Details of the disposable bodily fluid handling composite pad according to the present invention will be described hereunder on the basis of a menstruation napkin as one of typical embodiments of the invention. Construction of the menstruation napkin 1 shown by FIGS. 1 and 2 are common to Examples 1 through 7. FIG. 1 is a perspective view showing the menstruation napkin 1 as partially cutaway for convenience of illustration and FIG. 2 is a sectional view taken along the line II-II in FIG. 1. The menstruation napkin 1 has a longitudinal direction Y and a transverse direction X wherein the menstruation napkin 1 is relatively long in the longitudinal direction Y and has a width gradually reduced from longitudinally opposite ends toward a central region. The menstruation napkin 1 has the imaginary longitudinal center line P-P bisecting a width dimension in the transverse direction X and the imaginary transverse center line Q-Q bisecting a length dimension in the longitudinal direction Y. The menstruation napkin 1 is shaped substantially symmetric about the imaginary longitudinal center line P-P as well as about the imaginary transverse center line Q-Q.

The menstruation napkin 1 has an upper side facing the wearer's body and the opposite lower side facing the wearer's garment. The menstruation napkin 1 comprises two or more liquid-absorbent component pads laminated from the upper side facing the wearer's body to the lower side facing the wearer's garment. Specifically, the menstruation napkin 1 comprises a first component pad 10 defining the uppermost layer of the laminate, a second component pad 20 underlying the first component pad 10 and a third component pad 30 underlying the second component pad 20. These first, second and third component pads 10, 20, 30 respectively comprise upper liquid-absorbent sheets 11, 21, 31 and lower liquid-impervious sheets 12, 22, 32. The liquid-absorbent sheets 11, 21, 31 may be formed of spun lace fibrous non-woven fabric made of hydrophilic fiber such as cotton having a basis weight in a range of about 15 to 40 g/m², preferably in a range of about 20 to 35 g/m², more preferably of about 30 g/m². The liquid-impervious sheets 12, 22, 32 may be formed of plastic film having a basis weight in a range of about 10 to 25 g/m², preferably of about 18 g/m². The liquid-impervious sheets 12, 22, 32 preferably have breathability.

The lower side, i.e., the side facing the wearer's garment of the third component pad 30 is formed with joining means 40 such as adhesive which is, in turn, covered from below with a releasable sheet 50. The releasable sheet 50 may be peeled off from the third pad 30 to expose the joining means 40 by the intermediary of which the third pad 30 may be fastened to the wearer's panty.

The first, second and third component pads 10, 20, 30 and the releasable sheet 50 are same in shape as well as in size one to another and respective have the dimension in the transverse direction X gradually reduced from longitudinally opposite ends toward a central region defined by the imaginary transverse center line Q-Q. By dimensioning the component pads 10, 20, 30 in this manner, it is assured that these component pads are put in close contact with the wearer's body without irregular deformation of these component pads in the crotch region.

The first, second and third component pads 10, 20, 30 are formed along the peripheral edge 2 thereof with individual temporarily joined regions 13, 23, 33. Specifically, these individual temporarily joined regions 13, 23, 33 comprise a plurality depressions (debossings) arranged intermittently along the peripheral edge 2. The depressions are formed by collectively hot-pressing these first, second and third component pads 10, 20, 30 in the thickness direction so as to define the continuous linear temporarily joined region along which these component pads may be temporarily joined together. Each of the temporarily joined regions is formed continuously so that the respective depressions are contiguous one to another and defining the linear temporarily joined region or so that the respective depressions are arranged intermittently and collectively defining the substantially continuous linear temporarily joined region. The temporally joints between the temporally joining regions 13, 23, 33 are easily peeled off by pulling upward the respective component pads 10, 20, 30 so as to be spaced from one another.

Each of these first, second and third component pads 10, 20, 30 has a non-joined region 14, 24 or 34 along a part of its peripheral edge 2 where the temporarily joined region is not formed. In other words, these non-joined regions 14, 24, 34 are formed so as to partially interrupt the continuous temporarily joined regions 13, 23, 33, respectively. It should be appreciated that the position at which the non-joined region 14, 24 or 34 is formed is not limited to that in the embodiment as exemplarily shown by FIG. 1 but may be formed in the other various patterns. The respective component pads 10, 20, 30 may be formed with an indicator elements serving to indicate the positions of these non-joined regions 14, 24, 34.

### Example 1

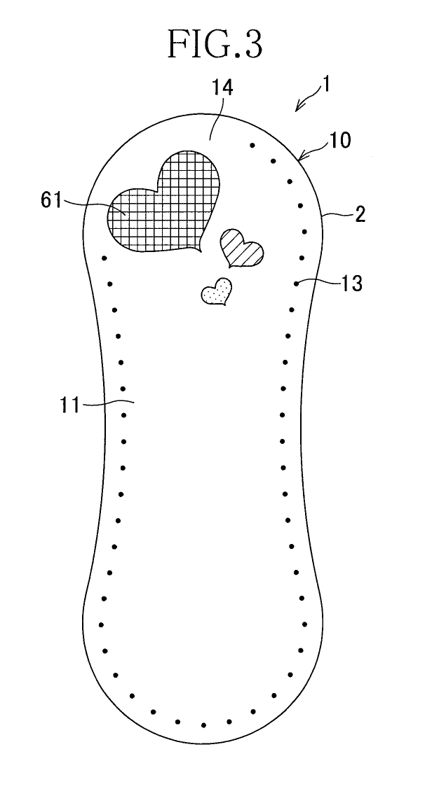

FIG. 3 shows Example 1 in which graphics are used as an indicator element 61. The indicator element 61 seen in FIG. 3 is visually recognized on the first component pad 10. The first component pad 10 is formed with the substantially continuous temporarily joined region 13 which is interrupted by the non-joined region 14 as shown in the upper area of FIG. 3 as viewed in the longitudinal direction Y. Heart marks as the indicator element 61 are provided in the non-joined region 14 so as to be visually recognized through the liquid-absorbent sheet 11. These heart marks facilitate the wearer to recognize the non-joined region 14 and to pinch the peripheral edge 2 in the vicinity of these heart marks with the fingers and thereby to peel off the first component pad 10.

While the first component pad 10 alone is illustrated in this Example 1, it will be understood that the second component pad 20 also is formed in the non-joined region 24 with the similar graphics. Thereby, the position of the second component pad 20 to be pinched by the wearer can be also easily recognized. While it is unnecessary to peel off the third component pad 30, the third component pad 30 also may be optionally provided with the similar heart marks. In this case, the number of the heart marks may be varied in the order of the first, second and third component pads 10, 20, 30 to make the wearer recognize what number component pad is being used. Obviously, the graphics are not limited to the heart marks and may be selected from various graphics such as snow crystal marks, flower marks or the like.

### Example 2

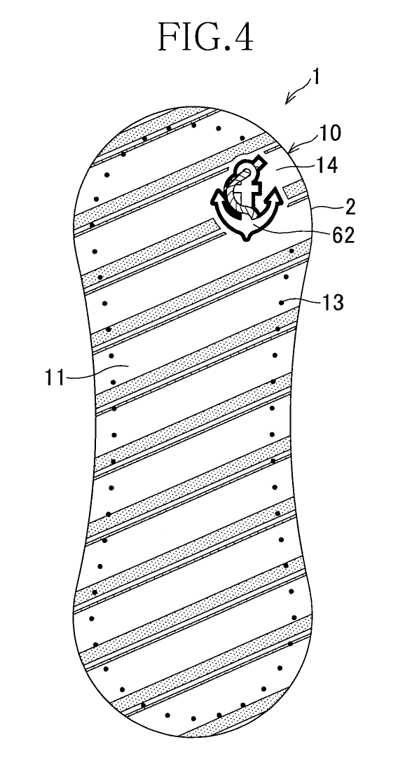

FIG. 4 is a view similar to FIG. 3, showing Example 2 wherein a graphic is used as an indicator element 62. The first component pad 10 is formed along the peripheral edge 2 with the temporarily joined regions 13 defining the substantially continuous temporarily joined line which is interrupted by non-joined region 14 as shown in the upper area of FIG. 4. In the non-joined region 14, an anchor mark is provided as the indicator element 62. In this Example, in addition to the anchor mark as the indicator element 62, another pattern is formed so that both the anchor mark and the pattern may be seen through the liquid-absorbent sheet 11. Specifically, the first component pad 10 is formed overall with a striped pattern. Also when the first component pad 10 is formed with the striped pattern, the non-joined region 14 can be easily recognized by forming the first component pad 10 with the graphic distinguished from such striped pattern.

As in the case of Example 1, the second component pad 20 and the third component pad 30 also may be provided with the same indicator element 62 and pattern as those provided on the first component pad 10. In this case, the pattern and the color thereof on the second component pad 20 and the third component pad 30 may be differentiated form those on the first component pad 10 to assure that the wearer can identify the respective component pads one from another.

### Example 3

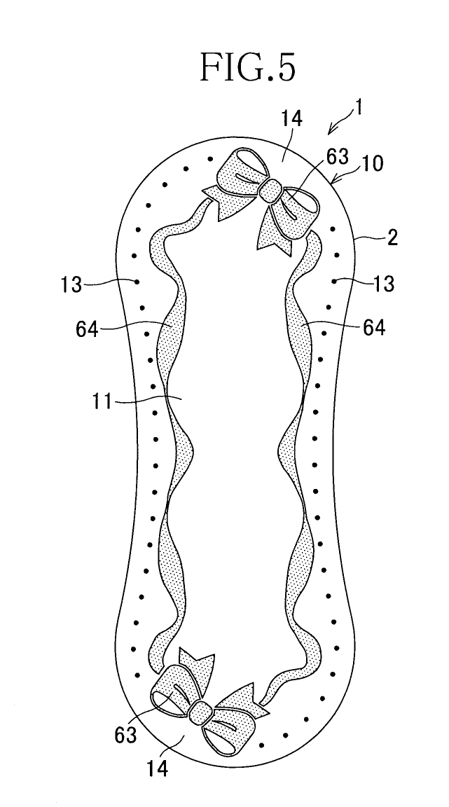

FIG. 5 is a view similar to FIG. 3, showing indicator elements 63 used in Example 3. The first component pad 10 is formed along transversely opposite side edges with two temporarily joined regions 13, respectively, and two non-joined regions 14 are defined between these two temporarily joined regions 13, as shown in the upper and lower areas of FIG. 5. In the respective non-joined regions 14, graphics of knotted ribbon are displayed as the indicator elements 63 and, along the respective temporarily joined regions 13, graphics 64 of ribbon pieces without knots are displayed.

The presence of graphics 64 displayed along the respective temporarily joined regions 13 assists the wearer to recognize the presence of these temporarily joined regions 13. The indicator elements 63 cooperate with the graphics 64 to clarify differences between the non-joined regions 14 and the temporarily joined regions 13 so that the wearer can recognize the non-joined regions 14 more easily.

It should be appreciated that it is not essential for the respective graphics 64 to precisely fall on the associated temporarily joined regions 13 and the respective graphics 64 may be sufficiently near by the associated temporarily joined regions 13 to distinguish the temporarily joined regions 13 from the non-joined regions 14. It is also possible to use flower pieces as the indicator elements 63, for example, and to use graphics of ivy leaves as the graphics 64 so that the indicator elements 63 and the graphics 64 may be meaningfully associated one with another. The indicator elements 63 and the graphics 64 used for the first component pad 10 may be used for the second component pad 20 and the third component pad 30 also.

### Example 4

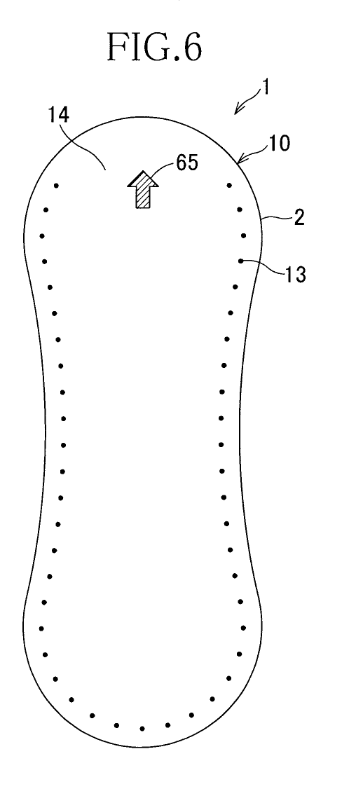

FIG. 6 illustrates Example 4 wherein a symbol is used as an indicator element 65. The first component pad 10 is formed along the peripheral edge 2 with the substantially continuous temporarily joined region 13 which is interrupted by the non-joined region 14 as shown in the upper area of FIG. 6 as viewed in the longitudinal direction Y. In the vicinity of the non-joined region 14, an arrowhead symbol is displayed as the indicator element 65 serving to indicate the presence of the non-joined region 14.

The indicator element 65 is not limited to the arrowhead symbol and various symbols other than the arrowhead symbol may be used as the indicator element 65 so far as the non-joined region can be thereby indicated. For example, the non-joined region 14 can be indicated with graphics of fingers or triangle. As in the case of Example 1, the same indicator element 62 and pattern or graphic as those provided on the first component pad 10 may be provided on the second component pad 20 and the third component pad 30 also.

### Example 5

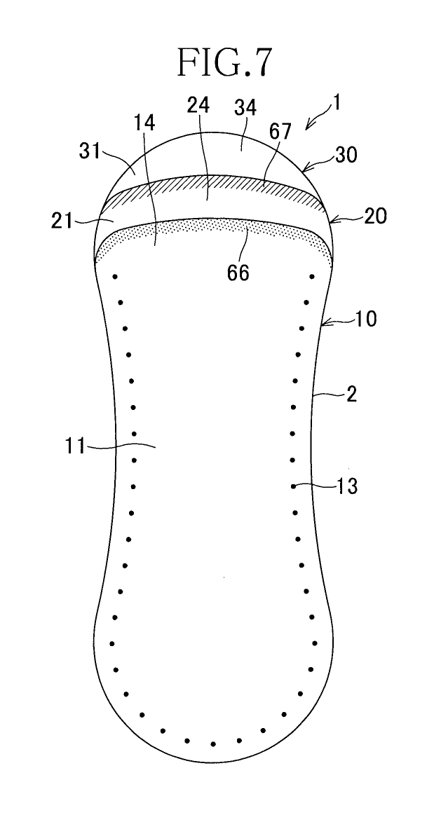

FIG. 7 illustrates Example 5 wherein the indicator elements are colored so as to be distinguished from other regions. The first, second and third component pads 10, 20, 30 are formed along the peripheral edge 2 with the substantially continuous temporarily joined regions 13, 23, 33 which are, in turn, interrupted by the non-joined regions 14, 24, 34, respectively, as shown in the upper area of FIG. 7. As shown in the upper area of FIG. 7, one of the opposite ends of the second pad 20 recedes from the corresponding end of the third component pad 30 so that the second component pad 20 has an area smaller than that of the third component pad 30. In other words, the non-joined region 34 of the third component pad 30 extends outward from underneath and beyond the non-joined region 24 of the second component pad 20. Such non-joined region 24 may be colored differently from the other regions to define an indicator element 67.

As shown in the upper area of FIG. 7, one of the opposite ends of first pad 10 recedes from the corresponding end of the second component pad 20 so that the first component pad 10 has an area smaller than that of the second component pad 20. In other words, the non-joined region 24 of the second component pad 20 extends outward from underneath and beyond the non-joined region 14 of the first component pad 10. Such non-joined region 14 is colored differently from the other regions so as to define an indicator element 66. More specifically, this indicator element 66 is colored differently from the indicator element 67 of the second component pad 20.

In the menstruation napkin 1 as has been heretofore described, the non-joined region 24 of the second component pad 20 extends outward from the non-joined region 14 of the first component pad 10 and the non-joined region 34 of the third component pad 30 extends outward from the non-joined region 24 of the second component pad 20. Such arrangement facilitates the wearer to understand the manner in which these three component pads are laminated one upon another and to localize the non-joined regions.

### Example 6

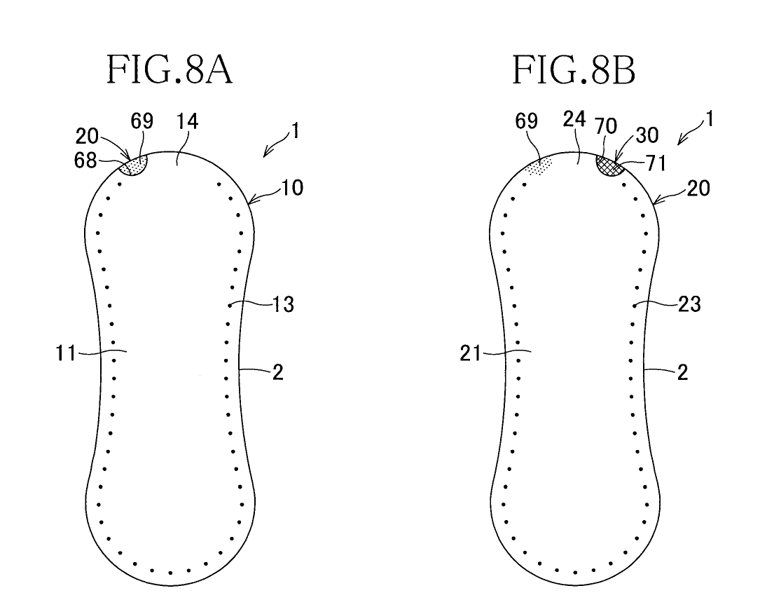

FIG. 8 illustrates Example 6 wherein the pad is formed with cutouts as indicator elements. Specifically, FIG. 8A illustrates the state of the composite pad 1 in which the first, second and third component pads 10, 20, 30 are laminated one upon another and FIG. 8B illustrates the state of the composite pad 1 after the first component pad 10 has been peeled off from the state as illustrated by FIG. 8A. The first, second and third component pads 10, 20, 30 are respectively formed along the peripheral edge 2 with the substantially continuous temporarily joined regions 13, 23, 33 which are respectively interrupted by the non-joined regions 14, 24, 34 formed in the upper regions of these component pads, respectively, as viewed in FIG. 8.

As shown in upper area of FIG. 8A, the first component pad 10 has the non-joined region 14 partially cut away to define the cutout 68. The region of the second component pad 20 corresponding to the cutout 68 is colored differently from the other regions to define a colored region 69. In this way, the colored region 69 can be visually recognized through the cutout 68, i.e., the cutout 68 and the colored region 69 cooperate with each other to define the indicator element.

The second component pad 20 is exposed as the first component pad 10 is peeled off from the composite pad 1 and this second component pad 20 has its non-joined region 24 partially cut away so as to define a cutout 70. The region of the third component pad 30 corresponding to the cutout 70 is colored differently from the other regions to define a colored region 71. With such arrangement, the colored region 71 is visually recognized through the cutout 70, i.e., the colored region 71 and the cutout 70 cooperate with each other to form the indicator element. The region 71 may be colored differently from the colored region 69.

The composite pad 1 according to Example 6 is formed with the cutout regions 68, 70 through which the colored regions 69, 71 of the underlying component pad can be visually recognized. Such arrangement facilitates the non-joined regions 14, 24 to be recognized. It should be understood that the colored regions 69, 71 are not essential features and may be eliminated without departing the scope of the invention. Even if none of these colored regions 69, 71 is provided, the cutouts 68, 70 effectively assist the wearer to recognize the non-joined regions 14, 24.

### Example 7

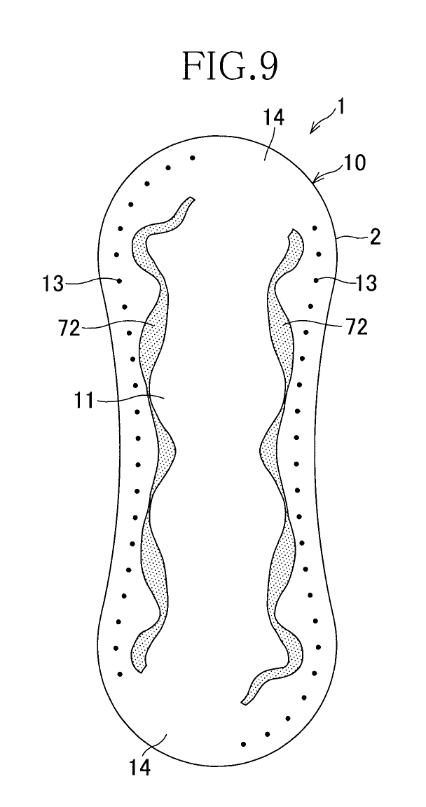

FIG. 9 is a view similar to FIG. 5, illustrating an indicator element 72 used by Example 7. The first component pad 10 is formed along transversely opposite side edges with two temporarily joined regions 13, respectively, and two non-joined regions 14 are defined between these two temporarily joined regions 13 as shown in upper and lower areas of FIG. 9. In this Example, the regions including none of the indicator elements can be recognized as the non-joined regions 14 merely by displaying the indicator elements 72 in the respective temporarily joined regions 13. Graphics of knot-less ribbon pieces are displayed as the indicator elements 72.

It should be appreciated that it is not essential for the indicator elements 72 to precisely fall on the associated temporarily joined regions 13 and the respective graphics 64 may be sufficiently near by the associated temporarily joined regions 13 to distinguish the temporarily joined regions 13 from the non-joined regions 14. It is possible to use graphics of ivy leaves as the indicator elements 72, for example. These indicator elements 72 preferably comprise graphics which can be drawn so as to be contiguous one to another so that the regions in which the graphics are interrupted can be recognized as the non-joined regions 14.

In the respective Examples, the indicator element may be formed not only by printing or the like technique but also by locally embossing the surfaces of the respective component pads. When the printing or the like technique is employed, the indicator element may be printed directly on the liquid-absorbent sheet or on the liquid-impervious sheet so that the indicator element can be visually recognized through the liquid-absorbent sheet. It is also possible to use letter, numeral or the like or a combination of such letter or the like and graphic, symbol, pattern or the like as the indicator element. While the respective Examples have been described on the assumption that each of them is the trilaminar composite pad, the invention is not limited to the trilaminar composite pad and the number of the component pads may be optional so far as the number of the component pads is two or more. However, it is important to temporarily join the respective component pads along the temporarily joined regions 13, 23, 33 without unintentionally being peeled off these component pads from the composite pad put on the wearer's body.

In the lowermost third component pad 30, fibrous non-woven fabric such as spunbond/melt blown/spunbond (SMS) non-woven fabric may be additionally laminated on the lower surface of the third component pad 30 and the joining means 40 is provided on this fibrous non-woven fabric. The non-woven fabric additionally laminated on the third component pad 30 serves as a reinforcing member preventing the third component pad 30 from being damaged in the course of peeling off the third component pad 30 from the wearer's garment such as panty for the purpose of exchanging the composite pad 1. By using the fibrous non-woven fabric as the reinforcing member, stiffness of the liquid-impervious sheet 32 constituting the third component pad 30 can be reduced and thereby feeling of discomfort which might otherwise be experienced by the wearer during use of the composite pad 1 can be alleviated.

1. A disposable bodily fluid handling composite pad having a longitudinal direction, a transverse direction, an upper side facing a wearer's body and a lower side opposite to said upper side, and comprising component pads each including an upper liquid-absorbent sheet and a lower liquid-impervious sheet wherein two or more of said component pads are laminated one on another, said disposable bodily fluid handling composite pad being characterized in that:
said two or more component pads respectively include temporarily joined regions along which said component pads are temporarily joined to each other so as to be peeled off from other component pads and non-joined regions; and
each of said component pads are formed with indicator elements adapted to be visually recognized through said liquid-absorbent sheet and said indicator elements are formed in said non-joined regions defined along peripheral edges of said component pads and adapted to indicate at least partially said non-joined regions.

2. The disposable bodily fluid handling composite pad defined by claim 1, wherein said temporally joined regions are formed substantially in continuous fashion along peripheral edges of said component pads and said non-joined regions are defined along partial segments of said peripheral edges so as to interrupt said substantially continuous temporally joined regions.

3. The disposable bodily fluid handling composite pad defined by claim 1, wherein said indicator element comprises at least any one of letter, symbol and graphic displayed in said non-joined regions.

4. The disposable bodily fluid handling composite pad defined by claim 1, wherein said indicator element comprises at least any one of letter, symbol and graphic serving to indicate said non-joined regions.

5. The disposable bodily fluid handling composite pad defined by claim 1, wherein said indicator element is defined by coloring of said non-joined regions.

6. The disposable bodily fluid handling composite pad defined by claim 1, wherein said indicator element comprises cutouts formed by partially cutting away said component pads.
